(19) United States
(12) Patent Application Publication
(10) Pub. No.: US 2008/0200402 A1
(43) Pub. Date: Aug. 21, 2008

(54) Use Of Fumagillin And The Derivatives Thereof To Increase The Bioavilability Of The Macrocyclic Lactones

(75) Inventors: Michel Alvinerie, Colomiers (FR); Jacques Dupuy, Toulouse (FR); Anne Lespine, Toulouse (FR); Jean-Francois Sutra, Saint Lys (FR)

Correspondence Address: Young & Thompson, 745 S. 23rd Street., Second Floor, Arlington, VA, 22202, US

(73) Assignee: INSTITUT NATIONAL DE LA RECHERCHE AGRONOMIQUE, Paris Cedex 07 (FR)

(21) Appl. No.: 11/917,031

(22) PCT Filed: Jun. 8, 2006
(86) PCT No.: PCT/FR2006/001297

§ 371 Date: Mar. 10, 2008

(30) Foreign Application Priority Data
Jun. 8, 2005 (FR) 0505829

(51) Int. Cl. A61K 31/336; A61K 31/7048; A61P 35/00; A61P 33/00

(52) U.S. Cl. 514/27; 514/475

(57) ABSTRACT

The invention relates to the use of fumagillin and the analog derivatives thereof, as inhibitors of cellular transporters, such as ABC transporters, and especially of P-glycoprotein, in order to increase the bioavailability of active ingredients that can be used in the treatment of pathologies such as cancers or parasitic diseases, and especially in order to increase the bioavailability of the macrocyclic lactones.

The invention relates to the use of fumagillin, and its analogous derivatives, as inhibitors of cellular transporters, such as ABC transporters, and more particularly of P-glycoprotein, in order to increase the bioavailability of active ingredients which can be used in the treatment of pathologies such as cancers or parasitic illnesses, and in particular in order to increase the bioavailability of macrocyclic lactones.

Macrocyclic lactones or ML, such as avermectins and milbemycins, are antiparasitic molecules for veterinary use which are very powerful (active against endo and ectoparasites, long remanence, low toxicity).

The avermectins are compounds of the following general formula:

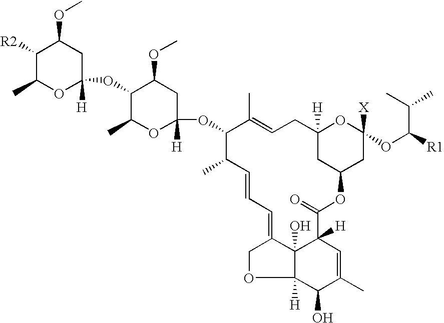

Ivermectin B₁ₐ is the compound of previous formula with X≡—CH₂CH₂— and R₁═CH(CH₃)CH₂CH₃;

Abamectin B₁ₐ is the compound of previous formula with X≡—CH═CH— and R₁═CH(CH₃)CH₂CH₃;

Doramectin is the compound of previous formula with X≡—CH═CH— and R₁=cyclohexyl;

Eprinomectin is the compound of previous formula with X≡—CH═CH—, R₁═CH(CH₃)CH₂CH₃, and R₂—NHCOCH₃.

Another avermectin, selamectin, is the compound of the following formula:

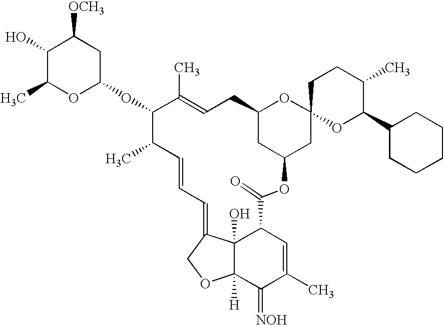

The milbemycins constitute another family of ML. Among the milbemycin, nemadectin is the compound of the following formula:

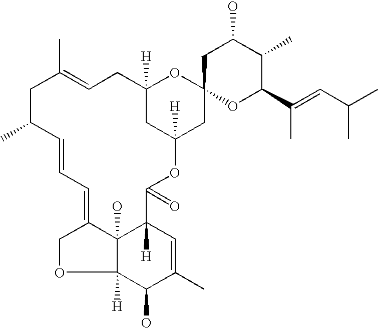

and moxidectin is the compound of the following formula:

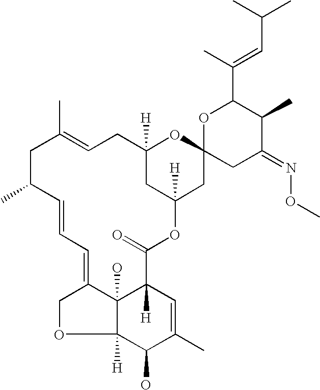

The advantages of ML are at the origin of a significant use in numerous mammals (bovines, ovines, caprines, pigs, horses, dogs and cats). Ivermectin is also used in human medicine for the treatment of Onchocerciasis (de Silva et al., 1997). The situation of macrocyclic lactones in the veterinary medicament market is economically important.

However, due to unsuitable usage (use of doses or of unrecommended administration routes and/or use in species for which no market authorization exists), phenomena of parasitic resistance have appeared in numerous species. Because no novel molecules have been developed, it is of prime importance to optimize the use of macrocyclic lactones (ML) by respecting that they are used safely. The antiparasitic activity of these compounds is directly linked to the concentration of active ingredient in the animal organism. Therefore, optimization of their effectiveness is via the increase in the quantity of medicament in the host animal after administration.

Physiological and pharmacological methods have been used for increasing the bioavailability of ML such as the reduction in the food intake in sheep (Ali and Hennessy, 1996), fasting in horses (Alvinerie et al., 2000), the co-administration of medicaments (Lifschitz et al., 2002) or of natural compound (Dupuy et al., 2003).

Among the numerous factors which can modulate the bioavailability of ML, P-glycoprotein or Pgp is recognized as one of the major factors both on cells (Dupuy et al., 2001b), in the entire animal (Alvinerie et al., 1999; Dupuy et al., 2003; Lifschitz et al., 2002) and in parasites (Xu et al., 1998). In fact, the membrane transporter of the family of “ATP binding cassette transporters”, involved in the mechanisms of polychemoresistance (multidrug resistances or MDR), control of the active efflux of numerous compounds including ivermectin and moxidectin. This Pgp is present at the level of the blood-brain barrier where it protects the central nervous system from the neurotoxicity of ivermectin (Roulet et al., 2003; Schinkel et al., 1994). The involvement of Pgp in the elimination of ML by biliary and intestinal routes, which are major elimination routes, has been demonstrated (Laffont et al., 2002).

The hepatocytes of rats in primary culture were used to evaluate the ability of different compounds to increase the intracellular quantity of ¹⁴C moxidectin. In this model, verapamil (recognized inhibitor of Pgp) or quercetin (a natural flavonoid that interferes with Pgp) significantly increases the quantity of ¹⁴C moxidectin in the hepatocytes of rats (Dupuy et al., 2001b; Dupuy et al., 2003). Similarly, ketoconazole significantly increases the intracellular quantity of ¹⁴C moxidectin, an effect which is linked to its concomitant inhibitory action on Pgp's and the cytochromes P450, two systems which are present in the hepatocytes.

Moreover, the modulation of Pgp is also useful for increasing the bioavailability of different active ingredients of ML. The modulation of Pgp is for example useful within the framework of the treatment of cancer, in order to increase the bioavailability of anticancerous active ingredients.

Therefore, several agents that modulate Pgp have recently been used to treat acute myeloblastic leukaemias (AML) and acute lymphoblastic leukaemias (ALL) in humans (Roos 2004), as summarized in Table 1 below:

TABLE 1
use of compounds which modulate the activity of Pgp in order
to treat acute myeloblastic leukaemias (AML) and acute lymphoblastic
leukaemias (ALL) in humans (Roos 2004)

| | | compounds modulating |
|---|---|---|
| Type of cancer | Treatment | the Pgp activity |
| LAM | Daunorubicin | Cyclosporin |
| LAM | Etoposide, mitoxantrone | Cyclosporin |
| LAM | Etoposide, daunorubicin | Valspodar (PSC 833) |
| LAM, LAL | Mitoxantrone | Quinine |

Surprisingly, the inventors have discovered that fumagillin and its derivatives make it possible to increase the bioavailability of ML, which provides a novel technical solution to the problem described above.

Fumagillin is produced by the fungus Aspergillus fumigatus, which is active in vivo on the microsporidia of bees and in vitro on the spores of Enterocytozoon Bieneusi (Fumidil B CEVA Sante Animale). In humans, fumagillin has been used for forty years to treat intestinal amoebiasis and is currently prescribed as a local application for keratoconjonctivis caused by microsporidia. Moreover, fumagillin and its analogues are angiogenesis inhibitors via the inhibition of endothelial cellular proliferation (Pyun et al., 2004). Due to their antiangiogenic properties, these compounds are used in human medicine for the treatment of cancers.

A main purpose of the invention is to provide compositions making it possible to increase the bioavailability of active ingredients in the human and animal organism, and thus to improve existing treatments, in particular within the framework of parasitic or cancerous diseases.

A more particular purpose of the invention is to provide compositions making it possible to increase the bioavailability of macrocyclic lactones and anti-tumoral agents. Principally the invention relates to the use of at least one compound of general formula (I) which follows:

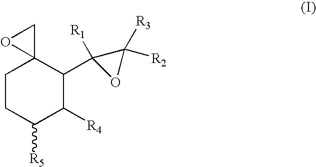

in which:
R₁ is H or a linear or branched C₁₋₈ alkyl;
R₂ is H, a C₁₋₄ alkyl, an aryl, an aryl C₁₋₄ alkyl, a cycloalkyl, a cycloalkyl C₁₋₄ alkyl, or an alkenyl group with 1 to 10 carbon atoms such as a CH₂R₆ group, in which R₆ is a 2-methyl-1-propenyl or an isobutyl optionally substituted by a hydroxyl, amino, (C₁₋₃ alkyl)-amino or di(C₁₋₃ alkyl)-amino group;
R₃ is an H atom, a C₁₋₄ alkyl, or a C₅₋₈ aryl which is optionally substituted by one or more halogens, such as F, Cl, 1, Br, a C₁₋₄ alkoxyl or a C₁₋₄ alkyl
R₄ is an H atom, an OH or a C₁₋₄ alkoxyl;
R₅ is of the form OR₇, in which case the bond [symbol] represents a single bond, or R₅ is of the form

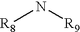

in which case the [symbol] bond represents a bond in α or β position;
R₇ is chosen from the group composed of:
   the H atom,
   a C₁₋₁₀ alkanoyl or alkenoyl group, saturated or unsaturated, which can be substituted in particular by one to three substituents chosen from amino, (C₁₋₆ alkyl)-amino, di-(C₁₋₆ alkyl)-amino, nitro, halogeno, hydroxy, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms, the C₁₋₆ alkyls, the C₁₋₆ alkoxys, the halogenated and nitro alkyls), and the aromatic heterocyclic groups,
   an aroyl group which can be substituted by a halogen atom or by a C₂₋₆ alkyl, amino, hydroxy, C₁₋₆ alkoxy, cyano, carbamyl or carboxyl,
   a heterocyl-carbonyl which can be substituted by a halogen atom or by a C₂₋₆ alkyl, amino, hydroxy, C₁₋₆ alkoxy, cyano, carbamyl or carboxyl,
   a carbamyl, which can be substituted by one or two substituents chosen from the C₁₋₆ alkyl groups, themselves being able to be substituted by a mono- or di-(C₁₋₆ alkyl)-amino, C₁₋₆ alkanoyl, chloroacetyl, dichloroacetyl, trichloroacetyl, (C₁₋₆ alkoxy) carbonyl-methyl, carboxy-methyl, phenyl optionally substituted (by one to five substituents chosen from the halogen atoms, the C₁₋₆ alkyls, the C₁₋₆ alkoxys, the halogenated and nitro alkyls), naphthyl or benzoyl group,
   a C₁₋₁₀ alkyl with a linear or branched chain, which can optionally be epoxidated and/or substituted in particular by one to three substituents chosen from amino, (C₁₋₆ alkyl)-amino, di-(C₁₋₆ alkyl)-amino, nitro, halogeno, hydroxy, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms, the C₁₋₆ alkyls, the C₁₋₆ alkoxys, the halogenated and nitro alkyls), and the aromatic heterocyclic groups,
   a C₁₋₁₀ alkenyl with a linear or branched chain,
   a C₁₋₁₀ alkynyl with a linear or branched chain,
   a cycloaliphatic hydrocarbon residue,
   a (cyclic amine)-carbonyl,
   a benzene-sulphonyl, which can be optionally substituted by one to three substituents chosen from the C₁₋₆ alkyls and the halogen atoms,
   a C₁₋₁₀ alkyl-sulphonyl, which can be optionally substituted by one to three substituents chosen from amino, (C₁₋₆ alkyl)-amino, di-(C₁₋₆ alkyl)-amino, nitro, halogeno, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms, the C₁₋₆ alkyls, the C₁₋₆ alkoxys, the halogenated and nitro alkyls), and the aromatic heterocyclic groups,
   a sulphamyl, which can be optionally substituted by one or two substituents chosen from the C₁₋₆ alkyls and a phenyl optionally substituted (by one to five substituents chosen from the halogen atoms, the C₁₋₆ alkyls, the C₁₋₆ alkoxys, the halogenated and nitro alkyls),
an alkoxy-carbonyl, which can be optionally substituted by one to three substituents chosen from amino, (C₁₋₆ alkyl)-amino, di-(C₁₋₆ alkyl)-amino, nitro, halogeno, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms, the C₁₋₆ alkyls, the C₁₋₆ alkoxys, the halogenated and nitro alkyls), and the aromatic heterocyclic groups,
   a phenoxycarbonyl, which can be optionally substituted by one to three substituents chosen from the halogen atoms and the C₁₋₆ alkyls,
   C(O)—NH—C(O)—CH₂—Cl;
R₈ and R₉ each represent an H atom, an optionally substituted hydrocarbon group or an optionally substituted acyl group, or R₈ and R₉ can constitute a ring together with the adjacent nitrogen atom;
   as adjuvant for the preparation of a medicament intended for increasing the bioavailability of active ingredients, in particular of antiparasitic or anticancerous active ingredients, and therefore to potentiate their effects, these active ingredients being capable of being recognized and binding to cellular transporters in order to be transported out of these cells without being able to reach their intracellular therapeutic target, said transporters being present in the cells of the human or animal organism to which said active ingredients are administered, and, if appropriate, into the cells of parasites against which these active ingredients are administered.

More particularly, the subject of the invention is the above-mentioned use of the compounds of formula (I) chosen from the following compounds of formula (Ia):

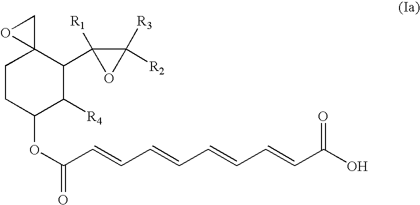

in which R₁, R₂, R₃, and R₄ are as defined above.

More particularly, the invention relates to the above-mentioned use of the compounds of formula (I) chosen from the following compounds of formula (Ib):

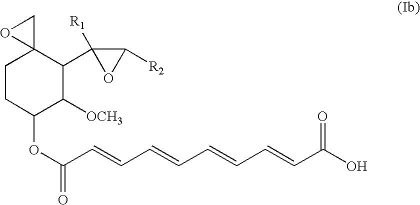

in which:
R₁ is H or a linear or branched C₁₋₈ alkyl;
R₂ is H, a C₁₋₄ alkyl, or an alkenyl group with 1 to 10 carbon atoms such as a CH₂R₆ group in which R₆ is a 2-methyl-1-propenyl.

More particularly, the invention relates to the above-mentioned use of the compound of formula (I) above corresponding to fumagillin of the following formula (II):

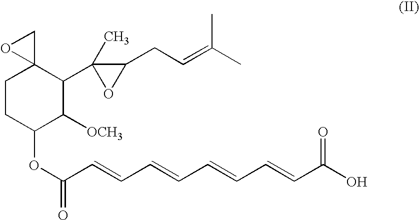

By “adjuvant” is meant a compound which is part of a pharmaceutical composition for a medicament, in order to “potentiate”, in other words to intensify and/or enable and/or to speed up the action of the base compound, said base compound also being called in this case “active ingredient”.

By “bioavailability” of an active ingredient is meant the quantity of active ingredient effectively present in a human or animal organism, and/or the quantity of active ingredient effectively present in a specific part of a human or animal organism, in particular in one or more specific organ(s) of a human or animal organism, and in particular in a subset of cells of one or more specific type(s) of a human or animal organism. The “bioavailability” can also indicate the proportion of the active ingredient administered to a human or animal organism which is effectively active or capable of activity in said organism.

More particularly, the subject of the invention is the above-mentioned use of the compounds of formula (I) above, and in particular of the compounds of formula (Ia), (Ib), and fumagillin of formula (II), for the preparation of a medicament intended to increase the bioavailability of active ingredients, more particularly of antiparasitic active ingredients, capable of being recognized and of binding to dependant ATP cellular transporters, also called ABC transporters (ATP Binding Cassette) or ATP-binding sequence transporters, these transporters being described in particular in Dean et al. (2001), Genome Research, 11: 1156-1166, and Dean et al. (2001), Journal of Lipid Research, 42:1007-1017.

More particularly, the invention relates to the above-mentioned use of the compounds of formula (I) above, and in particular of the compounds of formula (Ia), (Ib), and fumagillin of formula (II), for the preparation of a medicament intended to increase the bioavailability of active ingredients, more particularly of antiparasitic active ingredients, capable of being recognized and of binding to ABC transporters chosen from P-glycoprotein (Pgp, also called ABCB1), the ABCC transporters (ABCCl to 8, also called MRP1 to 8), or the ABC G2 transporters.

More particularly, the subject of the invention is the above-mentioned use of the compounds of formula (I) above, and in particular of the compounds of formula (Ia), (Ib), and fumagillin of formula (II), as inhibitors of the transport function of cellular transporters by interaction between these compounds and these transporters, for the preparation of a medicament intended to increase the bioavailability of active ingredients, more particularly of antiparasitic active ingredients, capable of being recognized and of binding to these transporters.

More particularly, the invention relates to the above-mentioned use of the compounds of formula (I) above, and in particular of the compounds of formula (Ia), (Ib), and fumagillin of formula (II), as inhibitors of the transport function of Pgp by interaction between these compounds and Pgp, for the preparation of a medicament intended to increase the bioavailability of active ingredients, more particularly of antiparasitic active ingredients, capable of being recognized and of binding to Pgp.

Advantageously, the above-mentioned compounds of formula (I) are used as adjuvants for the preparation of a medicament intended to increase the bioavailability of antiparasitic or anticancerous active ingredients chosen from the substrates of cellular transporters, and more particularly from the substrates of the ABC transporters defined above, in particular Pgp, within the framework of the treatment of parasitic or cancerous pathologies.

Advantageously the above-mentioned compounds of formula (I) used as adjuvants for the preparation of a medicament intended to increase the bioavailability of antiparasitic or anticancerous active ingredients within the framework of the treatment of parasitic or cancerous pathologies are chosen from the compounds of formula (Ia), (Ib), and fumagillin of formula (II).

Preferably, the above-mentioned compound of formula (I) used as adjuvant for the preparation of a medicament intended for the treatment of parasitic or cancerous pathologies is fumagillin of formula (II).

Advantageously, the above-mentioned use of the compounds of formula (I) above, and more particularly of the compounds of formula (Ia), (Ib), and fumagillin of formula (II), is characterized in that the antiparasitic active ingredients are chosen from the macrocyclic lactones, such as the avermectins and the milbemycins.

Advantageously, the above-mentioned use of the compounds of formula (I) above, and more particularly of the compounds of formula (Ia), (Ib), and fumagillin of formula (II), is characterized in that the antiparasitic active ingredients are chosen from the avermectins, such as ivermectin, abamectin, doramectin, eprinomectin or selamectin, within the framework of the treatment of parasitic, endoparasitic and ectoparasitic diseases.

Advantageously, the above-mentioned use of the compounds of formula (I) above, and more particularly of the compounds of formula (Ia), (Ib), and fumagillin of formula (II), is characterized in that the antiparasitic active ingredients are chosen from the milbemycins, such as moxidectin or nemadectin within the framework of the treatment of parasitic, endoparasitic or ectoparasitic diseases.

Endoparasitic diseases concern internal parasitic infections, while ectoparasitic diseases concern external parasitic infections.

Among parasitic diseases advantageously treated by ML and therefore falling within the framework of the invention, are in particular:
gastrointestinal strongylosis (adult and L3 or L4 larva): Haemonchus, Ostertagia, Trichostrongylus, Cooperia, Oesophagostonum, Nematodirus, Bunostonum;
pulmonary strongylosis: Dictyocaulus viviparus;
hypodermosis (all larval stages): Hypoderma bovis and lineatum;
sarcoptic and psoroptic mange;
phthiriasis;
filariosis;
onchocerciasis.

Equally advantageously, the above-mentioned use of the compounds of formula (I) above, and more particularly of the compounds of formula (Ia), (Ib), and fumagillin of formula (II), is characterized in that the anticancerous active ingredients are chosen from the substrates of cellular transporters, and more particularly substrates of the ABC transporters defined above, in particular Pgp, within the framework of the treatment of cancers, and more particularly of cancers resistant to chemotherapies.

By “cancers resistant to chemotherapies” is meant cancers which in response to chemical treatments overexpress cellular transporters, such as ABC-transporters, in particular the Pgp. By effluxing the active ingredient out of the cell, these transporters reduce or neutralize the expected therapeutic effect.

The anticancerous active ingredients which are the substrates of the above-mentioned cellular transporters, and more particularly Pgp, are in particular:
anthracycline-type antitumoral antibiotics, and in particular:
   daunorubicin and doxorubicin (used in the treatment of acute leukaemias, chronic myeloid leukaemias with acute transformation, Hodgkin's and non-Hodgkin's lymphomas),
   mitomycin C (used in the treatment of cancers of the breast, the stomach, the Esophagus, the bladder),
   mitoxantrone (used in the treatment of myeloid or acute lymphocytic leukaemia, cancer of the breast, the prostate, the ovary),
   adriamycin (used in the treatment of acute leukaemias, chronic myeloid leukaemias with acute transformation, Hodgkin's and non-Hodgkin's lymphomas),
   actinomycin-D (used in the same case as adriamycin),
taxanes, and in particular:
   docetaxel (used in the treatment of lymphomas, of cancer of the breast, the esophagus, the stomach, the bladder, the prostate, the uterus),
   paclitaxel (used in the treatment of cancer of the ovary, the lung, Kaposi's sarcoma linked with AIDS),
alkaloids, and in particular:
   vinblastine (used in the treatment of cancer of the breast, the bladder, the testicles and lymphomas),
   vincristine (used in the treatment of leukaemia, lymphomas, sarcomas, cancer of the lung, the uterus, the brain),
the epipodophyllotoxins, and in particular:
   etoposide (used in the treatment of cancer of the testicles and certain types of cancer of the lung),
   irinotecan (used in the treatment of colorectal cancer),
   teniposide (used in the treatment of cancer of the lung, the brain, the breast),
   topotecan.

Another aspect of the invention relates to a pharmaceutical composition characterized in that it comprises at least one compound of the formula (I) as defined above in combination with one or more active ingredients capable of being recognized and binding to the above-mentioned cellular transporters, and more particularly to the ABC transporters defined above, in particular to Pgp, to be transported out of cells of the human or animal organism.

More particularly, the subject of the invention is a pharmaceutical composition as defined above, comprising at least one compound of the formula (I) chosen from the compounds of formula (Ia), (Ib) or (II) defined above.

According to a preferred embodiment, said pharmaceutical composition is characterized in that the active ingredients, in combination with a compound of formula (I) as defined above, and more particularly with a compound of formula (Ia), (Ib), or fumagillin of formula (II), are antiparasitic or anticancerous active ingredients.

According to a more particular embodiment, said pharmaceutical composition is characterized in that it comprises at least one compound of the formula (I) as defined above, and more particularly a compound of formula (Ia), (Ib), or fumagillin of formula (II), in combination with antiparasitic active ingredients chosen from the macrocyclic lactones, such as the avermectins and the milbemycins.

According to a particularly preferred embodiment, said pharmaceutical composition is characterized in that the antiparasitic active ingredients are chosen from avermectins, such as ivermectin, abamectin, doramectin, eprinomectin or selamectin.

According to another particularly preferred embodiment, said pharmaceutical composition is characterized in that the antiparasitic active ingredients are chosen from milbemycins, such as moxidectin or nemadectin.

Advantageously, the above-mentioned pharmaceutical composition is characterized in that it contains at least one compound of the formula (I) as defined above, and more particularly a compound of formula (Ia), (Ib), or fumagillin of formula (II), at a dosage suitable for a daily administration of approximately 0.2 to approximately 2 mg/kg.

Advantageously, the above-mentioned pharmaceutical composition is characterized in that the antiparasitic active ingredient, and the compound of formula (I) as defined above, and more particularly the compound of formula (Ia), (Ib), or fumagillin of formula (II), are present in a ratio by weight comprised between approximately 1:1 and approximately 1:100, in particular between approximately 1:1 and approximately 1:20.

In general the compound of formula (I) as defined above, and more particularly the compound of formula (Ia), (Ib), or fumagillin of formula (II), must in fact be dosed in excess with respect to the active ingredient, because its affinity for the above-mentioned cellular transporters, in particular Pgp, is lower than that of the active ingredient.

More particularly, the invention relates to a pharmaceutical composition as defined above, comprising the above-mentioned fumagillin of formula (II), in combination with one or more antiparasitic active ingredients as defined above.

According to another preferred embodiment, the above-mentioned pharmaceutical composition is characterized in that it comprises at least one compound of formula (I) as defined above in combination with anticancerous active ingredients chosen from the substrates of the above-mentioned cellular transporters, and more particularly from substrates of the ABC transporters defined above, in particular Pgp; preferably this pharmaceutical composition comprises at least one compound of the formula (I) chosen from the compounds of formula (Ia), (Ib) or (II) defined above; this composition is characterized in a particularly advantageous manner in that it contains at least one compound of the formula (I), (Ia), (Ib) or (II) defined above at a dosage appropriate for a daily administration of approximately 0.2 to approximately 2 mg/kg; and in a yet more advantageous manner, this pharmaceutical composition is characterized in that the anticancerous active ingredient and the compound of formula (I), (Ia), (Ib) or (11) defined above, are present in a ratio by weight comprised between approximately 1:1 and approximately 1:100 and in particular between approximately 1:1 and approximately 1:20.

More particularly, the subject of the invention is a pharmaceutical composition comprising at least one compound of the formula (I) as defined above, and more particularly a compound of formula (I), (Ia), (Ib), or fumagillin of formula (II), in combination with at least one anthracycline-type antitumoral antibiotic and/or a taxane and/or an alkaloid and/or an epipodophyllotoxin as mentioned above.

More particularly, the invention relates to a pharmaceutical composition as defined above, comprising the above-mentioned fumagillin of formula (II), in combination with one or more anticancerous active ingredients as defined above.

In their various embodiments, the pharmaceutical compositions according to the invention are moreover advantageously characterized in that they are in a form which can be administered by a parenteral or oral route.

Another subject of the invention relates to combination products for a use which is simultaneous, separated or spread out over time, in therapy, in particular antiparasitic or anticancerous, using an active ingredient capable of being recognized and binding to the above-mentioned cellular transporters, and more particularly to the ABC transporters defined above, in particular to Pgp, to be transported out of the cells of the human or animal organism, characterized in that they contain at least one active ingredient as defined above, and at least one compound of the formula (I) as defined above, and more particularly fumagillin in of formula (II).

Advantageously, the compound of formula (I) in the combination products is chosen from the compounds of formula (Ia), (Ib) or (II) defined above.

Preferably, the combination products according to the invention are characterized in that they contain at least one active ingredient capable of being recognized and of binding to the above-mentioned cellular transporters, and more particularly to the ABC transporters defined above, in particular to Pgp, and at least one compound of the formula (I) as defined above, and more particularly a compound of formula (Ia), (Ib), or of fumagillin of formula (II), in a ratio by weight of approximately 1:1 to approximately 1:100 and in particular of approximately 1:1 to approximately 1:20.

More particularly, the combination products according to the invention, for a use which is simultaneous, separated or spread out over time, in antiparasitic therapy, are characterized in that they contain at least one antiparasitic active ingredient, and at least one compound of the formula (I) as defined above, and more particularly a compound of formula (Ia), (Ib), or fumagillin of formula (II).

Advantageously, the combination products according to the invention are characterized in that the antiparasitic active ingredients are chosen from macrocyclic lactones, such as avermectins and milbemycins.

In a more particular embodiment, the combination products according to the invention are characterized in that the antiparasitic active ingredients are chosen from avermectins, such as ivermectin, abamectin, doramectin, eprinomectin or selamectin, within the framework of the treatment of parasitic, endoparasitic and ectoparasitic diseases.

More particularly, said combination products are characterized in that the antiparasitic active ingredients are chosen from milbemycins, such as moxidectin or nemadectin, within the framework of the treatment of parasitic, endoparasitic or ectoparasitic diseases.

Advantageously, said combination products are characterized in that they contain at least one antiparasitic active ingredient, and at least one compound of the formula (I) as defined above, and more particularly fumagillin of formula (II), in a ratio by weight of approximately 1:1 to approximately 1:100 and in particular of approximately 1:1 to approximately 1:20.

More particularly, the subject of the invention is the combination products for a use which is simultaneous, separated or spread out over time, in antiparasitic therapy, characterized in that they contain at least one antiparasitic active ingredient as defined above, and fumagillin of formula (II) as mentioned above.

According to another advantageous embodiment, the combination products according to the invention, for a use which is simultaneous, separated or spread out over time, in cancer therapy, are characterized in that they contain at least one anticancerous active ingredient, and at least one compound of the formula (I) as defined above, and more particularly a compound of formula (Ia), (Ib), or fumagillin of formula (II).

Preferably, said combination products are characterized in that the anticancerous active ingredients are chosen from the substrates of the above-mentioned cellular transporters, and more particularly substrates of the ABC transporters defined above, in particular Pgp, within the framework of the treatment of cancers and more particularly of cancers resistant to chemotherapies, and are more particularly chosen from anthracycline-type antitumoral antibiotics, taxanes, alkaloids and epipodophyllotoxins as mentioned above.

In a particularly preferred manner, said combination products are characterized in that they contain at least one anticancerous active ingredient, and at least one compound of the formula (I) as defined above, and more particularly a compound of formula (Ia), (Ib), or fumagillin of formula (II), in a ratio by weight of approximately 1:1 to approximately 1:100 and in particular of approximately 1:1 to approximately 1:20.

More particularly, the subject of the invention is the combination products for a use which is simultaneous, separated or spread out over time, in anticancer therapy, characterized in that they contain at least one anticancerous active ingredient as defined above, and fumagillin of formula (II) as mentioned above.

### DESCRIPTION OF FIGURES

FIG. 1 shows the area under the curve (AUC) of ¹⁴C moxidectin after two separate treatments: in white, the control (moxidectin); in black: treatment with fumagillin. The y-axis is in μg.mL.h⁻¹. p<0.01 for the treatment with fumagillin (significantly different result from the control).

FIG. 2 shows the effect of different compounds on the accumulation of rhodamine 123 in LLCPK1 cells transfected with murine Pgp. On the x-axis: the concentration of the compound; on the y-axis, the percentage accumulation of Rho 123 relative to the control (Rho 123/protein content). White, ivermectin; uniform grey, valspodar; hatched, fumagillin.

FIG. 3 shows the accumulation of Rho 123 in Mdr1a LLC-PKI after treatment by fumagillin. On the x-axis, the concentration of fumagillin in μM; on the y-axis, the percentage effect relative to the valspodar effect (modelling according to the Hill model).

### EXPERIMENTAL PART

In this experimental part, firstly the capacity of fumagillin to increase the intracellular concentration of ¹⁴C moxidectin in rat hepatocytes is shown (Example 1). Its capacity to interfere with the Pgp function in the epithelial cells of a pig's kidney transfected with murine Pgp (Mdr Ia-LLCPK1) is then evaluated, as summarized in Example 2. The transport function of Pgp is evaluated by the intracellular accumulation of rhodamine 123, a known substrate of Pgp. This model is particularly suitable for detecting compounds interacting with Pgp (Hamada et al., 2003).

Chemical Compounds and Media

The standard solution of ¹⁴C moxidectin (radiopurity=98.2%, chemical purity>99%, specific activity=14.8 μCi/mg) was provided by Strong-Dodge Sante Animale (Tours, France). Fumagillin was provided by CEVA Sante Animale (Libourne, France). Valspodar (VSP) was kindly provided by Novartis (Basle, Switzerland). Dimethyl-sulphoxide (DMSO), sodium dodecyl sulphate (SDS), collagen, rhodamine 123 (Rho 123), trypsine-EDTA and ivermectin were purchased from Sigma Chimie (Saint-Quentin Fallavier, France). Medium 199, phosphate buffer saline (PBS10×), foetal calf serum, Hanks' buffer saline solution (HBSS) without phenol red, penicillin, streptomycin and geniticin (G418) originate from InVitrogen (Cergy Pontoise, France). The culture dishes are from Nunclon (Roskilde, Denmark), the culture flasks and 24-well culture plates from Sarstedt France (Orsay, France). The bicinchoninic acid kit originates from Interchim (Montlucon, France). Acetonitrile and methanol (RS quality for high-performance liquid chromatography) were purchased from Carlo Erba (Milan, Italy). The water used during this study was ultra-pure quality (MilliQ A10 device, Millipore ITS, Saint-Quentin, France).

Isolation of Hepatocytes, Culturing and Treatments

The isolation and culturing of rat hepatocytes has been described previously (Dupuy et al., 2001b). The hepatocytes are distributed into culture dishes and kept at 37° C. for 12 hours (oven 5% CO₂). The cells are cultured in the presence of 5 μM ¹⁴C moxidectin (control)+/−100 μM fumagillin. After 0, 6, 24, 48 and 72 h the incubations are stopped (n=3 for each treatment), the media collected and the hepatocytes harvested by mechanical disassociation in phosphate buffer saline (PBS 1X). The media and the hepatocytes are stored at −20° C. until analysis by high-performance liquid chromatography (HPLC).

Mdr1a-LLCPK1 and Intracellular Accumulation of Rhodamine 123 (Rho 123)

The cells transfected with the murine Pgp (Mdr1a-LLCPK1) were cultured in medium 199 supplemented with penicillin (100 units/ml) and streptomycin (100 μg/ml), 10% foetal calf serum and geniticin sulphate (G418, 400 μg/ml) as Pgp selection agent. The confluent cells are subcultured by trypsinization each week and the medium renewed twice weekly. They are kept at 37° C. in a controlled atmosphere at 5% CO₂. In order to monitor the transport function via Pgp in the Mdr1a-LLCPK1 cells, the intracellular accumulation of Rho¹²³ is measured. The Mdr1a-LLCPK1 cells are distributed onto (24-well) cell culture plates at a rate of 1.5.10⁵ cells/well. They are cultured for 48 hours at 37° C. to reach confluence in 1 ml of medium without G418. The medium is eliminated and the cells washed with 0.5 ml PBS 1X. The cells are cultured for 2 hours at 37° C. with 0.2 ml of HBSS medium containing 10 μM Rho 23 (HBSS/DMSO, 50/50, v/v) +/−5 μM VSP (in DMSO) +/−10 μM IVM (in DMSO) +/−1, 5, 10, 50 and 100 μM fumagillin (in DMSO). After 2 hours, the culture medium is eliminated, the cells washed with 0.5 ml PBS 1X to eliminate the excess Rho¹²³. The cells are lysed by the addition of 0.3 ml PBS 1X/0.5% sodium dodecyl sulphate (50/50, v/v) in each well. After 10 minutes at ambient temperature, 0.3 ml PBS 1X is added to each well then the total lysate (0.6 ml) is transferred to a 2-ml plastic tube and stored at −20° C. until it is analyzed by spectrofluorimetry.

Data Quantification and Analysis: ¹⁴C Moxidectin in the Cultured Hepatocytes

¹⁴C moxidectin is quantified in the medium and the hepatocytes by a HPLC technique coupled with an in-line radioactivity detection (Dupuy et al., 2001b). This technique allows ¹⁴C moxidectin and its main metabolite (C₂₉ monohydroxymethyl moxidectin) in rat hepatocytes to be detected and quantified. The radioactivity is measured by liquid scintillation counting (Kontron Beta V counter). The total initial radioactivity of the initial medium at 5 μM ¹⁴C moxidectin +/−100 μM fumagillin corresponds to the 100% value. Due to the concentration of moxidectin detected in the hepatocytes (ng.ml⁻¹) and to the initially introduced percentage of radioactivity, the time-concentration areas under the curve are calculated from the first to the last experimental point using the trapezoidal method (Gibaldi and Perrier, 1982).

Data Quantification and Analysis: Rho¹²³ Accumulation in Mdr1a-LLCPK1

The Rho¹²³ fluorescence is measured using a fluorimeter (Perkin Elmer LS50B, λₘₐₓ excitation=507 nm; λₘₐₓ emission=529 nm) then standardized with the protein content of each well (calorimetric detection reaction, BCA kit). The results are expressed in percentage accumulation of Rho 123 in the cells treated (VSP or IVM or fumagillin) relative to the control cells containing only Rho²³. In order to compare the different molecules, VSP is defined as the compound for which the Rho¹²³ accumulation is at a maximum and therefore corresponds to a 100% inhibition of Pgp. The results obtained were modelled according to the Hill model (Scientist software, Micromath research, Saint Louis, USA).

Statistical Analysis

The average and the standard deviation were determined for all the parameters studied. All the data were subjected to the Fischer test (PLSD Fischer test) via Statview software (Abacus Concept, Berkeley, USA). In all cases, a value of p<0.05 is considered significant.

### Example 1

### Rat Hepatocytes

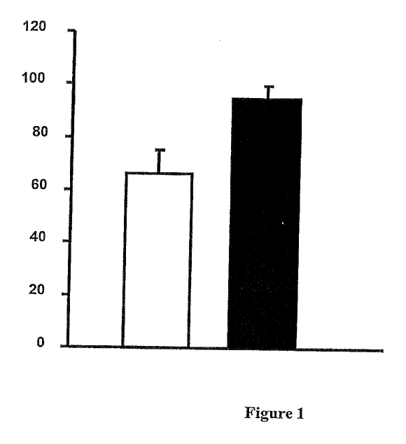

The viability of rat hepatocyte cultures (exclusion of trypan blue) is greater than 80% and no morphological change is observed during culture for 72 h, whatever the treatment. The main compound detected is moxidectin and the intracellular quantities are given in Table 2 below. The main metabolite corresponding to C₂₉ monohydroxymethyl already described during a previous study (Dupuy et al., 2001b) represents only 4% (maximum value) of the parent substance. Fumagillin significantly increases the quantity of intracellular moxidectin with a maximum at 6 h in the controls and after 24 h in the cells treated by fumagillin. The reduction in the concentration of moxidectin in the hepatocytes is more rapid in the controls (6 hours post-treatment) than those treated by fumagillin (24 hours post-treatment). The concentration of the main metabolite increases from 6 h to reach its maximum value 24 h after treatment and its production kinetics are not affected. The exposure of cells to moxidectin is quantified by the time-concentration area under the curve calculated over the course of the experiment (FIG. 1). Fumagillin significantly increases by 65% the quantity of moxidectin in the hepatocytes over a period of 72 h.

TABLE 2
quantity of ¹⁴C moxidectin in cultured rat hepatocytes after
treatment by moxidectin +/− fumagillin (100 μM)^{a}

| Culture duration | | |
|---|---|---|
| (hour) | Moxidectin | Moxidectin + fumagiliin |
| 0 | 4.5 ± 1.25 | 7.05 ± 0.24 |
| 6 | 72.95 ± 9.98 | 79.29 ± 1.01 |
| 24 | 52.60 ± 5.94 | 82.94 ± 14.97** |
| 48 | 22.19 ± 4.00 | 27.88 ± 2.67 |
| 72 | 11.98 ± 1.30 | 16.22 ± 1.21 |

^{a}The values represent the average ± standard deviation of 3 different culture dishes.
**significantly different to cells treated by moxidectin. P < 0.01

### Example 2

### Mdr1a-LLCPK1

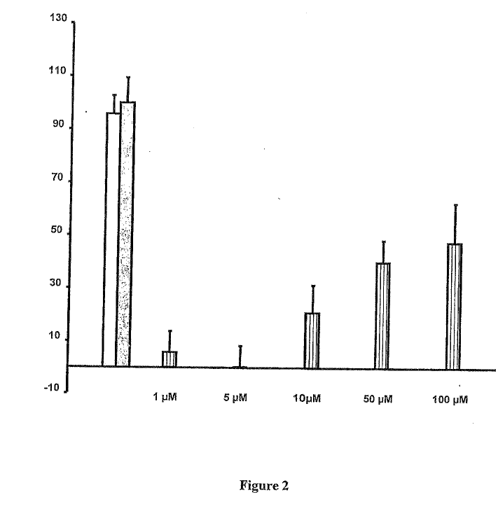

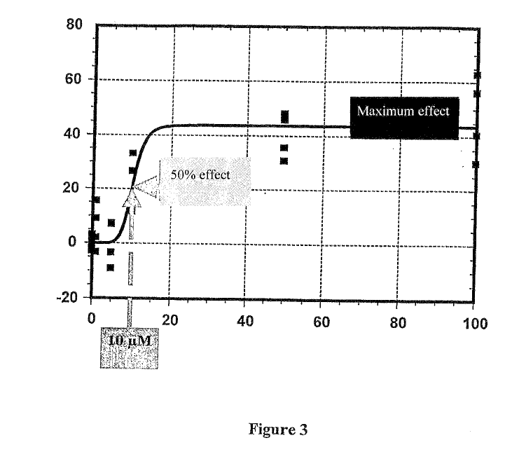

The intracellular accumulation of Rho¹²³ was monitored in order to evaluate the effect of fumagillin on Pgp activity in Mdr1a-LLCPK1 cells. This model was validated using 2 known compounds as agents which interfere with Pgp: IVM and VSP. The fluorescence results were standardized relative to the protein quantity. The effect induced by VSP (10 μM) is considered to be the maximum value (100%) for Rho 123 accumulation in the cells (FIG. 2). 5 μM IVM has an inhibitory power very close to that of VSP since it generates an effect representing 95% of the effect of VSP. Fumagillin (10 to 100 μM) allowed the quantity of intracellular Rho 123 to be increased. The results were then expressed in percentage accumulation relative to VSP and were modelled using the Hill model. A sigmoid curve was thus generated (FIG. 3). The maximum effect (Emax), defined as the maximum quantity of Rho¹²³ in the cells in the presence of fumagillin, is reached at a concentration of 50 μM of fumagillin and represents 43.7% of the effect obtained in the presence of VSP. EC50, the concentration necessary to reach 50% of the maximum effect, is obtained in the presence of 10 μM for fumagillin and represented 21.8% of the VSP effect.

### Discussion on the Experiments

In veterinary medicine, the ML remain the most effective antiparasitic compounds in particular on account of their broad action spectrum and their unique action mechanism. To ensure the lasting quality of these compounds, it is vital to optimize their use. One strategy consists of increasing the bioavailability of the compound, then the effectiveness of ML is directly linked to the presence of the medicament in the systemic circulation for a sufficient length of time. The pharmacological methods for the administration of chemical or natural compounds (Dupuy et al., 2003; Lifschitz et al., 2002) are mainly based on the involvement of active transporters such as Pgp which modulate the bioavailability of ML in animals and in parasites. Thanks to the use of Pgp inhibitors, the bioavailability of IVM in rats (Alvinerie et al., 1999) and moxidectin in sheep (Dupuy et al., 2003) was able to be increased. Moreover, the antiparasitic effectiveness was increased by the co-administration of ML and agents which interfere with Pgp in ivermectin- and moxidectin-resistant parasite strains. This shows that Pgp could play a role in the resistance of nematodes to ML (Molento and Prichard, 1999).

Previous studies have shown that primary-cultured rat hepatocytes represent a particularly useful tool for the study of the function of Pgp and Pgp/cytochrome P450 3A interactions (Dupuy et al., 2001b; Hirsch-Ernst et al., 2001). In fact, Pgp is expressed in hepatocytes and its expression is increased over time (Hirsch-Ernst et al., 1998). In this case, the capacity of fumagillin, a medicament used in veterinary and human medicine, to increase the quantity of intracellular moxidectin in rat hepatocytes was evaluated. Surprisingly, fumagillin induced an intracellular accumulation of moxidectin. Compared with the results previously obtained with this cellular model, the intracellular accumulation of moxidectin obtained with fumagillin (100 μM) is comparable to that obtained with quercetin with a maximum effect 24 hours after treatment (Dupuy et al., 2003). The reduction over time of the concentration of moxidectin in the hepatocytes (controls, treated with fumagillin), and also observed with quercetin, can be attributed to the activity of P450 cytochromes which is the basis for the production of metabolites which are rapidly expelled out of the hepatocytes. These results show that fumagillin can modulate the intracellular accumulation of moxidectin in the cellular system used here. The effect obtained with verapamil or quercetin definitely brings into play the involvement of Pgp in the accumulation of moxidectin in the hepatocytes model since these compounds are known to interfere with Pgp.

The influence of fumagillin on the intracellular accumulation of Rho 123 in Mdr1a-LLCPK1 cells (Schinkel et al., 1995) has also been studied. These cells overexpress murine Pgp and possess little or no other transporters of the same family (ABC transporters) or P450 cytochromes. A recent study showed that different ML allowed the effIux of Rho¹²³ and the accumulation of calceine in tumoral cells (Korystov et al., 2004). In our model, fumagillin allowed the quantity of Rho¹²³ to be increased in a dose-dependent manner, which implies an interaction with Pgp. The modelling of the effect of fumagillin allowed the percentage accumulation of Rho¹²³ to be correlated relative to the effect induced by a known inhibitor (VSP) of Pgp. The maximum effect obtained with 100 μM fumagillin corresponds to 43% of the VSP effect. But to date there is no data available on the interaction between fumagillin and Pgp or other ABC transporters. These results show that the increase in moxidectin observed in rat hepatocytes in the presence of fumagillin is associated with an inhibitory effect of this compound on the Pgp function.

For this reason, fuinagillin acquires a new interest in the field of veterinary medicine as a regulating agent for Pgp. Because of the emergence of resistance to macrocyclic lactones in numerous species and the absence of development of new powerful antiparasitic substances in the medium term, it is vital to develop strategies which aim to make the effectiveness of ML last. Fumagillin thus allows the effectiveness of ML vis-à-vis parasites to be increased by increasing the quantity of medicament within resistant parasites.

One such approach for potentializing the action of a compound effluxed by Pgp by the co-administration of a substance to reduce resistance phenomena is used in human cancer chemotherapy. Clinical trials are currently being conducted in patients suffering from cancer and developing resistance to anti-cancer drugs. The use of molecules which inhibit the Pgp function in conjunction with an anti-cancer drug allows the quantity of medicament in these patients to be increased in order to have an increased therapeutic effectiveness (List et al., 2001). Recently, it was demonstrated that avermectins were able to increase the quantity of anti-tumoral medicaments in cancer cells (Korystov et al., 2004). It can therefore be envisaged that macrocyclic lactones will be used in cancer chemotherapy.

### REFERENCES

Ali, D. N., and Hennessy, D. R. (1996). The effect of level of feed intake on the pharmacokinetic disposition and efficacy of ivermectin in sheep. J Vet Pharmacol Ther 19, 89-94.
Alvinerie, M., Dupuy, J., Eeckboutte, C., and Supra, J. F. (1999). Enhanced absorption of pour-on ivermectin formulation in rats by co-administration of the multidrug-resistant-reversing agent verapamil. Parasitol Res 85, 920-922.
Alvinerie, M., Supra, J. F., Cabezas, I., Rubilar, L., and Perez, R. (2000). Enhanced plasma availability of moxidectin in fasted horses. Journal of Equine Veterinary Science 20, 575-578.
de Silva, N., Guyatt, H., and Bundy, D. (1997). Anthelmintics. A comparative review of their clinical pharmacology. Drugs 53, 769-788.
Dupuy, J., Chartier, C., Supra, J. F., and Alvinerie, M. (2001a). Eprinomectin in dairy goats: dose influence on plasma levels and excretion in milk. Parasitol Res 87, 294-298.
Dupuy, J., Larrieu, G., Supra, J. F., Eeckhoutte, C., and Alvinerie, M. (2001b). Influence of verapamil on the efflux and metabolism of ¹⁴C moxidectin in cultured rat hepatocytes. J Vet Pharmacol Ther 24, 171-177.
Dupuy, J., Larrieu, G., Supra, J. F., Lespine, A., and Alvinerie, M. (2003). Enhancement of moxidectin bioavailability in lamb by a natural flavonoid: quercetin. Vet Parasitol 112, 337-347.
Gibaldi, M., and Perrier, D. (1982). Pharmacokinetics, 2nd (revised and expanded) edn (New York, Marcel Dekker Inc.).
Hamada, A., Miyano, H., Watanabe, H., and Saito, H. (2003). Interaction of imatinib mesilate with human P-glycoprotein. J Pharmacol Exp Ther 307, 824-828.
Hirsch-Ernst, K. I., Ziemann, C., Foth, H., Kozian, D., Schmitz-Salue, C., and Kahl, G. F. (1998). Induction of mdrlb mRNA and P-glycoprotein expression by tumor necrosis factor alpha in primary rat hepatocyte cultures. J Cell Physiol 176, 506-515.
Hirsch-Ernst, K. I., Ziemann, C., Rustenbeck, I., and Kahl, G. F. (2001). Inhibitors of mdr1-dependent transport activity delay accumulation of the mdr1 substrate rhodamine 123 in primary rat hepatocyte cultures. Toxicology 167, 47-57.
Korystov, Y. N., Ermakova, N. V., Kublik, L. N., Levitman, M., Shaposhnikova, V. V., Mosin, V. A., Drinyaev, V. A., Kruglyak, E. B., Novik, T. S., and Sterlina, T. S. (2004). Averinectins inhibit multidrug resistance of tumor cells. Eur J Pharmacol 493, 57-64.
Laffont, C. M., Toutain, P. L., Alvinerie, M., and Bousquet-Melou, A. (2002). Intestinal secretion is a major route for parent ivermectin elimination in the rat. Drug Metab Dispos 30, 626-630.
Lifschitz, A., Virkel, G., Sallovitz, J., Imperiale, F., P is, A., and Lanusse, C. (2002). Loperamide-induced enhancement of moxidectin availability in cattle. J Vet Pharmacol Ther 25, 111-120.
List, A. F., Kopecky, K. J., Willman, C. L., Head, D. R., Persons, D. L., Slovak, M. L., Dorr, R., Karanes, C., Hynes, H. E., Doroshow, J. H., et al. (2001). Benefit of Cyclosporin modulation of drug resistance in patients with poor-risk acute myeloid leukaemia: a Southwest Oncology Group study. Blood 98, 3212-3220.
Molento, M. B., and Prichard, R. K. (1999). Effects of the multidrug-resistance-reversing agents verapamil and CL 347.099 on the efficacy of ivermectin or moxidectin against unselected and drug-selected strains of Haemonchus contortus in jirds (Meriones unguiculatus). Parasitol Res 85, 1007-1011.
Roos D.D. (2004), Best Practice & Research Clinical Haematology, 17: 641-651.
Roulet, A., Puel, O., Gesta, S., Lepage, J. F., Drag, M., Soll, M., Alvinerie, M., and Pineau, T. (2003). MDR₁-deficient genotype Collie dogs hypersensitive to the P-glycoprotein substrate ivermectin. Eur J Pharmacol 460, 85-91.
Schinkel, A. H., Smit, J. J., van Tellingen, O., Beijnen, J. H., Wagenaar, E., van Deemter, L., Mol, C. A., van der Valk, M. A., Robanus-Maandag, E. C., te Riele, H. P., et al. (1994). Disruption of the mouse mdrla P-glycoprotein gene leads to a deficiency in the blood-brain barrier and to increased sensitivity to drugs. Cell 77, 491-502.
Schinkel, A. H., Wagenaar, E., van Deemter, I., Mol, C. A., and Borst, P. (1995). Absence of the mdrla P-Glycoprotein in mice affects tissue distribution and pharmacokinetics of dexamethasone, digoxin, and cyclosporin A. J Clin Invest 96, 1698-1705.
Xu, M., Molento, M., Blackhall, W., Ribeiro, P., Beech, R., and Prichard, R. (1998). Ivemmectin resistance in nematodes may be caused by alteration of P-glycoprotein homolog. MolBiochemParasitol 91, 327-335.

1-23. (canceled)

24. Method for increasing the bioavailability of active ingredients, and therefore to potentiate their effects, these active ingredients being capable of being recognized and binding to cellular transporters present in the cells of the human or animal organism to which said active ingredients are administered, and, if appropriate, into the cells of parasites against which these active ingredients are administered, in order to be transported out of these cells without being able to reach their intracellular therapeutic target, wherein compounds of formula (I) 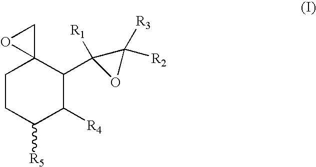
in which:
R₁ is H or a linear or branched C₁₋₈ alkyl;
R₂ is H, a C₁₋₄ alkyl, an aryl, an aryl C₁₋₄ alkyl, a cycloalkyl, a cycloalkyl C₁₋₄ alkyl, or an alkenyl group with 1 to 10 carbon atoms such as a CH₂R₆ group, in which R₆ is a 2-methyl-1-propenyl or an isobutyl optionally substituted by a hydroxyl, amino, (C₁₋₃ alkyl)-amino or di(C₁₋₃ alkyl)-amino group;
R₃ is an H atom, a C₁₋₄ alkyl, or a C₅₋₈ aryl which is optionally substituted by one or more halogens, such as F, Cl, I, Br, a C₁₋₄ alkoxyl or a C₁₋₄ alkyl
R₄ is an H atom, an OH or a C₁₋₄ alkoxyl;
R₅ is of the form OR₇, in which [symbol] case the bond represents a single 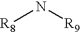
bond, or R₅
is of the form
in which case [symbol] the bond
represents a bond in α or β position;
R₇ is chosen from the group composed of:
the H atom,
a C₁₋₁₀ alkanoyl or alkenoyl group, saturated or unsaturated, which can be substituted in particular by one to three substituents chosen from amino, (C₁₋₆ alkyl)-amino, di-(C₁₋₆ alkyl)-amino, nitro, halogeno, hydroxy, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms,
the C₁₋₆ alkyls, the C₁₋₆ alkoxys, the halogenated and nitro alkyls), and the aromatic heterocyclic groups,
an aroyl group which can be substituted by a halogen atom or by a C₂₋₆ alkyl, amino, hydroxy, C₁₋₆ alkoxy, cyano, carbamyl or carboxyl,
a heterocyl-carbonyl which can be substituted by a halogen atom or by a C₂₋₆ alkyl, amino, hydroxy, C₁₋₆ alkoxy, cyano, carbamyl or carboxyl,
a carbamyl, which can be substituted by one or two substituents chosen from the C₁₋₆ alkyl groups, themselves being able to be substituted by a mono- or di-(C₁₋₆ alkyl)-amino, C₁₋₆ alkanoyl, chloroacetyl, dichloroacetyl, trichloroacetyl, (C₁₋₆ alkoxy)-carbonyl-methyl, carboxy-methyl, phenyl optionally substituted (by one to five substituents chosen from the halogen atoms, the C₁₋₆ alkyls, the C₁₋₆ alkoxys, the halogenated and nitro alkyls), naphthyl or benzoyl group,
a C₁₋₁₀ alkyl with a linear or branched chain, which can optionally be epoxidated and/or substituted in particular by one to three substituents chosen from amino, (C₁₋₆ alkyl)-amino, di-(C₁₋₁₆ alkyl)-amino, nitro, halogeno, hydroxy, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms, the C₁₋₆ alkyls, the C₁₋₆ alkoxys, the halogenated and nitro alkyls), and the aromatic heterocyclic groups,
a C₁₋₁₀ alkenyl with a linear or branched chain,
a C₁₋₁₀ alkynyl with a linear or branched chain,
a cycloaliphatic hydrocarbon residue,
a (cyclic amine)-carbonyl,
a benzene-sulphonyl, which can be optionally substituted by one to three substituents chosen from the C₁₋₆ alkyls and the halogen atoms,
a C₁₋₁₀ alkyl-sulphonyl, which can be optionally substituted by one to three substituents chosen from amino, (C₁₋₆ alkyl)-amino, di-(C₁₋₆ alkyl)-amino, nitro, halogeno, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms,
the C₁₋₆ alkyls, the C₁₋₆ alkoxys, the halogenated and nitro alkyls), and the aromatic heterocyclic groups,
a sulphamyl, which can be optionally substituted by one or two substituents chosen from the C₁₋₆ alkyls and a phenyl optionally substituted (by one to five substituents chosen from the halogen atoms, the C₁₋₆ alkyls,
the C₁₋₆ alkoxys, the halogenated and nitro alkyls),
an alkoxy-carbonyl, which can be optionally substituted by one to three substituents chosen from amino, (C₁₋₆ alkyl)-amino, di-(C₁₋₁₆ alkyl)-amino, nitro, halogeno, C₁₋₆ alkoxy, cyano, carbamyl, carboxyl, (C₁₋₆ alkoxy)-carbonyl, carboxy-(C₁₋₆ alkoxy), phenyl optionally substituted (by one to five substituents chosen from the halogen atoms,
the C₁₋₆ alkyls, the C₁₋₆ alkoxys, the halogenated and nitro alkyls), and the aromatic heterocyclic groups,
a phenoxycarbonyl, which can be optionally substituted by one to three substituents chosen from the halogen atoms and the C₁₋₆ alkyls,
C(O)—NH—C(O)—CH₂—Cl;
R₈ and R₉ each represent an H atom, an optionally substituted hydrocarbon group or an optionally substituted acyl group, or R₈ and R₉ can constitute a ring together with the adjacent nitrogen atom;
are used as adjuvant.

25. Method according to claim 24 wherein the compounds of formula (I) has the following formula (Ia): 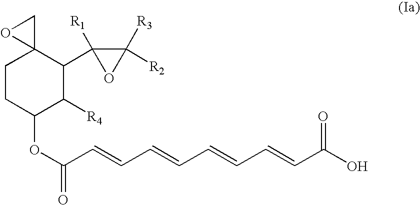
in which R₁, R₂, R₃, and R₄ are as previously defined.

26. Method according to claim 24 wherein the compounds of formula (I) has the following formula (Ib): 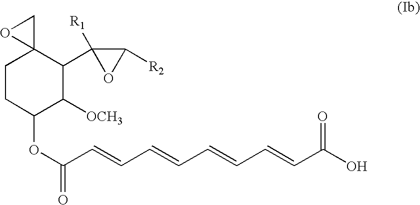
in which:
R₁ is H or a linear or branched C₁₋₈ alkyl;
R₂ is H, a C₁₋₄ alkyl, or an alkenyl group with 1 to 10 carbon atoms such as a CH₂R₆ group in which R₆ is a 2-methyl-1-propenyl.

27. Method according to claim 24 wherein the compound of formula (I) above corresponds to fumagillin of the following formula (II):

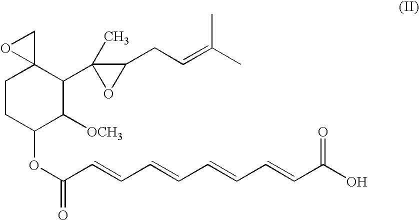

28. Method according to claim 24 to increase the bioavailability of active ingredients capable of being recognized and binding to dependant ATP cellular transporters, also called ABC transporters (ATP Binding Cassette) or ATP-binding sequence transporters.

29. Method according to claim 28 wherein the ABC transporters is chosen from P-glycoprotein, the ABCC transporters and the ABC G2 transporters.

30. Method according to claim 24 to increase the bioavailability of active ingredients capable of being recognized and binding to cellular transporters wherein the compound of formula (I) is used as inhibitor of the transport function of cellular transporters by interaction between these compounds and these transporters.

31. Method according to claim 24 to increase the bioavailability of active ingredients capable of being recognized and binding to Pgp wherein the compound of formula (I) is used as inhibitor of the transport function of Pgp by interaction between these compounds and Pgp.

32. Method according to claim 24 to increase the bioavailability of antiparasitic or anticancerous active ingredients within the framework of the treatment of parasitic or cancerous pathologies wherein the compound of formula (I) is chosen from the compounds of formula (Ia), (Ib) or (II).

33. Method according to claim 32, characterized in that the antiparasitic active ingredients are chosen from the macrocyclic lactones, within the framework of the treatment of parasitic, endoparasitic or ectoparasitic diseases.

34. Method according to claim 33 wherein the macrocyclic lactones are selected from avermectins and milbemycins.

35. Method according to claim 34 wherein the avermectin is selected from the group comprising ivermectin, abamectin, doramectin, eprinomectin and selamectin and the milbecyn is selected from the group comprising moxidectin and nemadectin.

36. Method according to claim 32, characterized in that the anticancerous active ingredients are chosen from the substrates of the cellular transporters within the framework of the treatment of cancers, and more particularly of cancers resistant to chemotherapies.

37. Pharmaceutical composition characterized in that it comprises at least one compound of formula (I) in combination with one or more active ingredients capable of being recognized and binding to cellular transporters.

38. Pharmaceutical composition according to claim 37, characterized in that it comprises at least one compound of formula (I) chosen from the compounds of formula (Ia), (Ib) or (II).

39. Pharmaceutical composition according to claim 38, characterized in that the active ingredients in combination with the compound of formula (I), (Ia), (Ib) or (II) are antiparasitic or anticancerous active ingredients.

40. Pharmaceutical composition according to claim 38, characterized in that it contains a compound of formula (I), (Ia), (Ib) or (II), at a dosage suitable for a daily administration of approximately 0.2 to approximately 2 mg/kg.

41. Pharmaceutical composition according to claim 39, characterized in that the active ingredient and the compound of formula (I), (Ia), (Ib) or (II) are present in a ratio by weight comprised between approximately 1:1 and approximately 1:100.

42. Pharmaceutical composition according to claim 41, wherein the ratio is between approximately 1:1 and approximately 1:20.

43. Pharmaceutical composition according to claim 37, characterized in that it comprises fumagillin of formula (II) in combination with one or more antiparasitic or anticancerous active ingredients.

44. Pharmaceutical composition according to claim 41, characterized in that it is in a form which can be administered by a parenteral or oral route.

45. Combination products for a use which is simultaneous, separated or spread out over time, in therapy, using an active ingredient capable of being recognized by cellular transporters characterized in that they contain at least one active ingredient as defined above, and a compound of the formula (I).

46. Combination products according to claim 45, characterized in that the compound of formula (I) is chosen from the compounds of formula (Ia), (Ib) or (II).

47. Combination products according to claim 45, for a use which is simultaneous, separated or spread out over time, in antiparasitic therapy, characterized in that they contain at least one active ingredient selected from macrocyclic lactones and a compound of formula (I), (Ia), (Ib) or (II).

48. Combination products according to claim 45, for a use which is simultaneous, separated or spread out over time, in anticancer therapy, characterized in that they contain at least one active ingredient selected from the cellular transporters and a compound of formula (I), (Ia), (Ib) or (II).

49. Combination products according to claim 48, characterized in that they contain at least one active ingredient capable of being recognized by cellular transporters, and a compound of formula (I), in a ratio by weight comprised between approximately 1:1 and approximately 1:100.

50. Combination products according to claim 49, wherein the ratio is between approximately 1:1 and approximately 1:20.

51. Combination products according to claim 45, characterized in that they contain at least one antiparasitic or anticancerous active ingredient and fumagillin of formula (II).
